(19) United States
(12) Patent Application Publication
(10) Pub. No.: US 2010/0145227 A1
(43) Pub. Date: Jun. 10, 2010

(54) LANCING DEVICE COMPRISING A TORSIONAL SPRING

(75) Inventor: Robert Wessel, Regensburg (DE)

Correspondence Address: BLACK LOWE & GRAHAM, PLLC, 701 FIFTH AVENUE, SUITE 4800, SEATTLE, WA, 98104, US

(73) Assignee: GERRESHEIMER WILDEN GMBH, Regensburg, DE (DE)

(21) Appl. No.: 12/529,905

(22) PCT Filed: Feb. 29, 2008
(86) PCT No.: PCT/EP08/52491

§ 371 Date: Jan. 25, 2010

(30) Foreign Application Priority Data
Mar. 5, 2007 (DE) 10 2007 011 002.4
May 24, 2007 (DE) 10 2007 024 183.8

(51) Int. Cl. A61B 5/151

(52) U.S. Cl. 600/583

(57) ABSTRACT

A lancing device for taking blood for medical examinations, comprising a base body, at least one needle which is arranged therein and which can be extended outwards with a tip end, and a manual actuating element for releasing a displacement movement of the needle, wherein a control curve section and a torsional spring that engages in the control curve section are arranged in a needle holding element, which at least partially encompasses the needle, in order to transform a radial movement of ends of the torsional spring into a translational displacement movement of the needle.

The invention relates to a lancing device for taking blood for medical examinations, comprising a base body (main body), at least one needle which is arranged therein and which can be extended outwards with a tip end, and a manual actuating element for releasing a displacement movement of the needle, according to the preamble of claim 1.

Lancet-type lancing devices for lancing a skin surface for the purpose of taking blood are known in various forms. Often such lancing devices require clinical specialists to apply these lancing devices in a largely pain-free manner.

DE 196 17 000 C1 discloses a device comprising a needle which serves for taking blood, wherein a hollow cannula is designed with a sharpened cutting edge. Such devices are usually not intended for single use and, due to the needle shape, are not always pain-free during use.

A system for taking blood comprising a blood receiving cylinder with a piston and a needle cylinder head with a needle holder which can be screwed onto an outer thread of the blood receiving cylinder is known from DE 44 43 276 A1. Such a system for taking blood is expressly intended for reuse and is designed so as to allow a germ-free reuse of this system. Accordingly, such systems are designed in an expensive manner and nevertheless harbour the risk of infections.

DE 297 18 67 U1 discloses a lancet for taking blood with a needle tip end protruding at the end region. The needle has an end region with the tip end which protrudes out from an axial end of a base body (main body) and which is embedded in a tubular attachment or connection region of a needle insertion head. The insertion head and the base body (main body) are formed in one piece. An axial pressure is exerted on this base body (main body) by means of a device (not shown) in order to allow the needle to penetrate to a certain depth into the body of the patient. The needle end region is then manually pulled out of the body of the patient. Such devices generally do not allow a pain-free lancing operation, since the handling by means of an additional pressure-exerting device allows only the insertion but not the removal of the needle.

U.S. Pat. No. 4,643,189 likewise discloses a lancing device which comprises a housing and a needle arranged therein which moves on a curved path. By moving along a certain curved path with the first end of a lever-type element, a needle tip which is connected to the second end of the lever-type element is moved towards the skin surface and then is removed again from the latter by way of a circular movement. Due to the movement of the needle on a circular path, such a construction is painful to the patient during use since the needle remains in the patient's skin for a relatively long time on account of the necessary circular movement, and an undesirably large cut may be obtained on the skin surface due to the circular movement carried out.

Accordingly, the object of the present invention is to provide a lancing device for taking blood for medical examinations, which allows an almost pain-free lancing movement even by the actual person being examined, while being of simple and inexpensive design, the lancing device being intended for single use.

This object is achieved by the features of claim 1.

One essential point of the invention lies in the fact that, in a lancing device for taking blood for medical examinations, comprising a base body (main body), at least one needle which is arranged therein and which can be extended outwards with a tip end, and a manual actuating element for releasing a displacement movement of the needle, a control curve section and a torsional spring that engages in the control curve section are arranged in a needle holding element, which at least partially encompasses the needle, in order to transform a radial movement of ends of the torsional spring into a forward and backward displacement movement of the needle. Due to the arrangement of a torsional spring, which may be of spiral shape with ends protruding perpendicular to the spiral plane, a space-saving and simple design of a single-use lancing aid or lancing device is obtained, which moreover always reliably provides the same penetration depth and profile path of the needle being displaced, regardless of the handling by the user. Due to the needle being displaced in the forward and backward direction, this allows a brief lancing operation which, due to the spring function, can be carried out almost without any pain and regardless of the handling by the user.

According to a first embodiment, the needle holding element is arranged such that it can be displaced together with the needle within the base body (main body) so that, during a desired displacement movement of the needle, the needle holding element is also displaced, the needle being arranged in a stationary manner relative to the needle holding element.

Alternatively, according to a further embodiment, the needle holding element can be arranged in a stationary manner in the base body (main body) and the needle can be displaced relative to the needle holding element and the base body (main body). This requires that the needle itself is displaced in the forward and backward direction and along the control curve section (guiding curve section) by means of the torsional spring both relative to the needle holding element and relative to the base body (main body).

The control curve section is arranged in a groove-like manner in the region of an end of the needle holding element opposite the tip end of the needle, wherein the first end of the torsional spring is mounted such that it can slide in the groove-like control curve section and the second end of the torsional spring is arranged in a stationary manner in the base body (main body). This allows in a simple manner the interaction of the needle and of the groove-like control curve section, wherein, due to the arrangement of the control curve section at the end of the needle holding element opposite the needle tip, the risk that the actual needle will be disadvantageously influenced in terms of its displacement movement by the torsional spring and the rotational movement thereof is ruled out on account of the spatial separation.

The groove-like control curve section and/or a groove located behind or in front of the latter in the base body (main body) preferably has a first region, the profile of which allows an arresting of the first end of the pretensioned torsional spring. It moreover has a second region, the profile of which is essentially V-shaped in an identical or similar form, or may be arc-shaped. As soon as a release lever arranged on the manual actuating element for releasing the first end of the torsional spring from its arrested position in the first region of the control curve section carries out a pivoting movement, the end of the spring is released from the first region and enters the second region. According to the first embodiment, this second region has a profile which includes a maximum point pointing towards the end of the displaceable needle holding element, i.e. for example represents an inverted V when the needle tip is pointing downwards.

Alternatively, according to the second embodiment, the second V-shaped or arc-shaped profiled region may have a maximum point pointing towards the tip end of the needle within the needle holding element arranged in a stationary manner in the base body (main body). In this case, the needle holding element is not displaceable within the base body (main body) and requires a displacement of the needle relative to the needle holding element, which is the reason why the maximum point or the lowest point within an arc-shaped path or a V-shaped path points towards the needle tip. This corresponds for example to a V-shape with the V open at the top when the needle tip is arranged at the bottom.

According to the first embodiment, the first end of the torsional spring, in the case of a displaceable needle holding element, at the same time engages in a groove which runs perpendicular to the direction of displacement of the needle holding element and which is arranged within the base body (main body) or an element connected in a stationary manner therewith. This allows a predefined position of the spring end relative to the base body (main body), while the needle holding element is moved forwards and backwards due to the control curve section traveled by the spring end. Alternatively, instead of being arranged within the base body (main body), the groove running perpendicular to the direction of displacement of the needle holding element may be arranged within the needle holding element, while the V-shaped groove is formed within the base body (main body). The desired displacement movement of the needle holding element relative to the base body (main body) is thus obtained. Of course, the V-shaped groove may also be arc-shaped.

The base body (main body) is preferably composed of two parts in such a way that a first part has the needle holding element arranged thereon and the torsional spring and a second part represents at least a portion of a housing. In this case, the first and the second part can be displaced relative to one another in an arresting manner, so that the penetration depth of the needle tip, which depends on the distance between the undersides of the first and second part, can be determined by a different displacement position of the first and second parts relative to one another. In this way, an individual setting of the penetration depth of the needle tip is possible in a simple manner, wherein this can be made dependent for example on the quantity of blood to be taken.

Further advantageous embodiments will emerge from the dependent claims.

Advantages and expedient features can be found in the following description in conjunction with the drawing, in which:

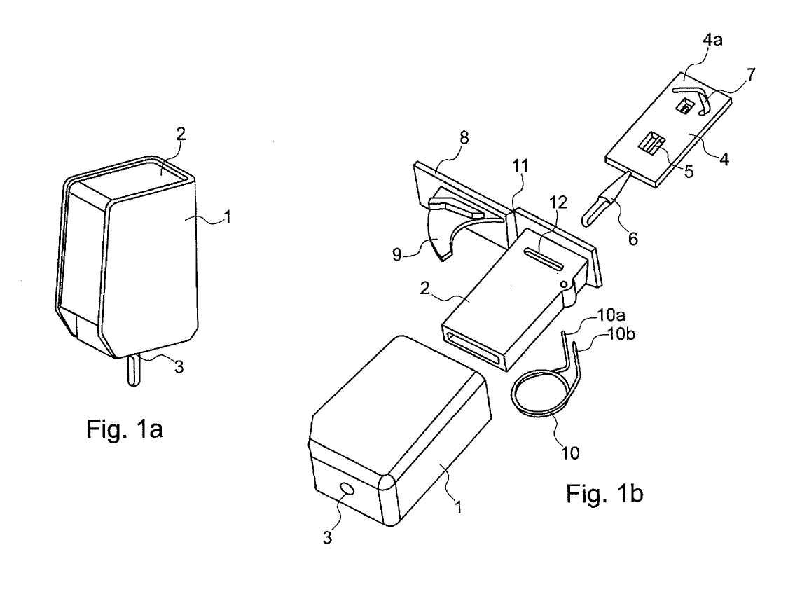

FIG. 1a shows a lancing device in a perspective view and in the assembled state according to a first embodiment of the invention;

FIG. 1b shows a lancing device according to the first embodiment of the invention in a perspective and disassembled view;

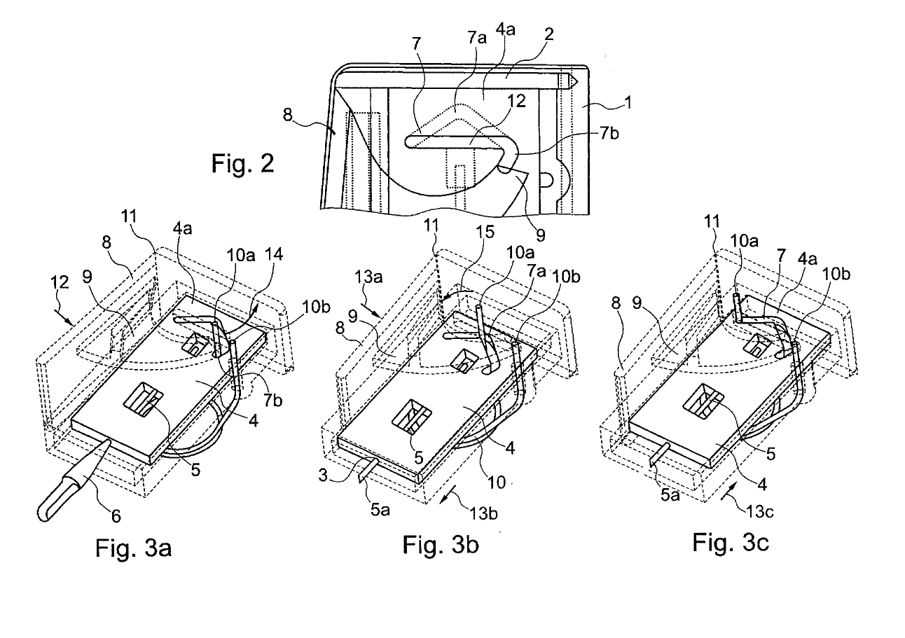

FIG. 2 shows a detail in partial cross-section of the curve profile of a control curve section of the lancing device according to the first embodiment of the invention;

FIGS. 3a-3c show in a perspective and partially open view the mode of operation of the lancing device according to the first embodiment of the invention in individual steps;

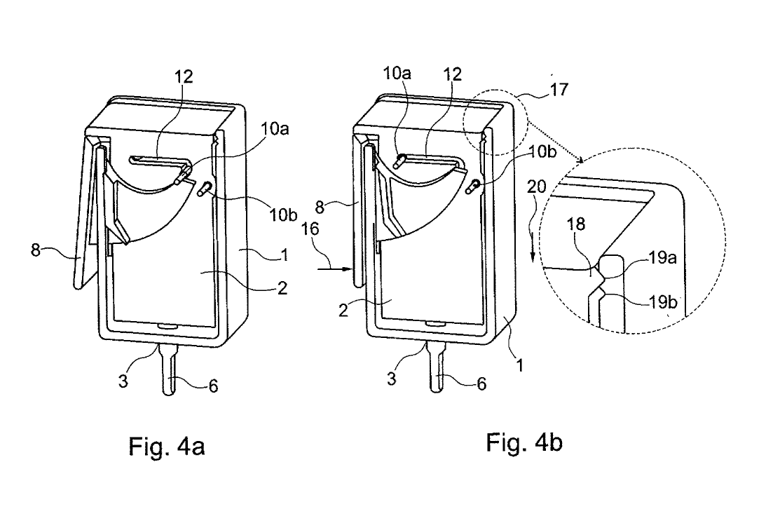

FIGS. 4a, 4b show a partially open and perspective view of the lancing device before and after a lancing operation has taken place, according to the first embodiment of the invention;

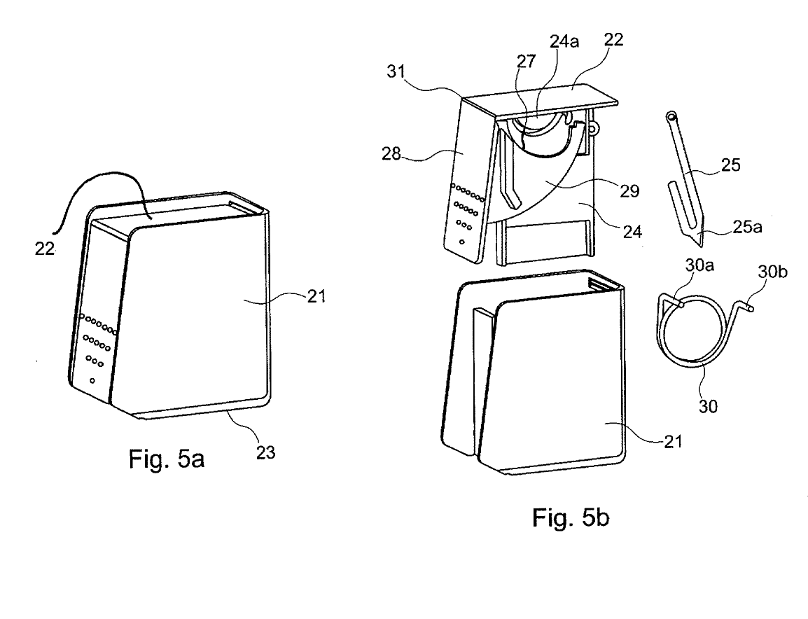

FIG. 5a shows a perspective view of a lancing device according to a second embodiment of the invention in the assembled state;

FIG. 5b shows the lancing device according to the second embodiment of the invention in a perspective and disassembled view;

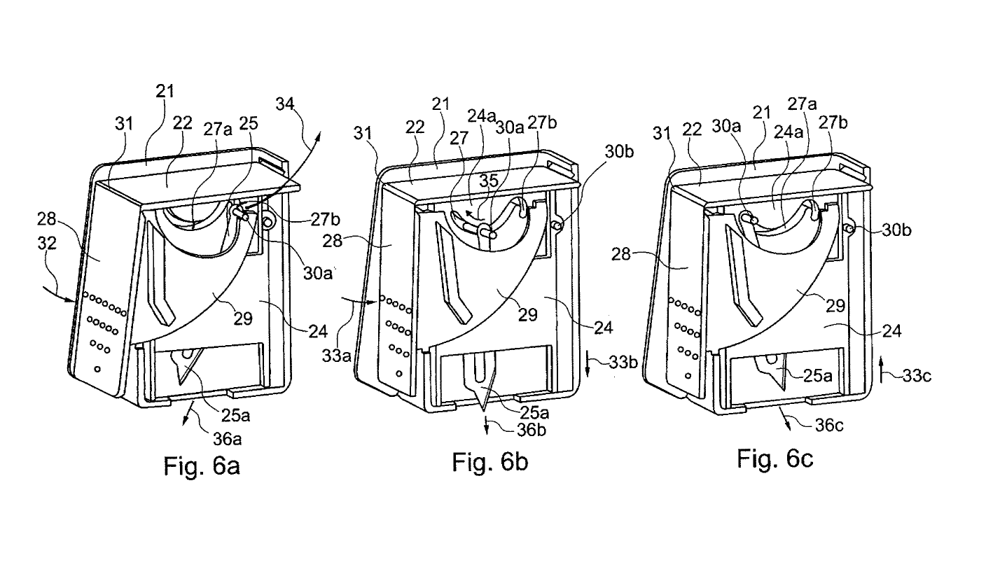

FIGS. 6a-6c show a perspective view of the lancing device according to the second embodiment of the invention in a partially open view showing individual functional steps during a lancing operation;

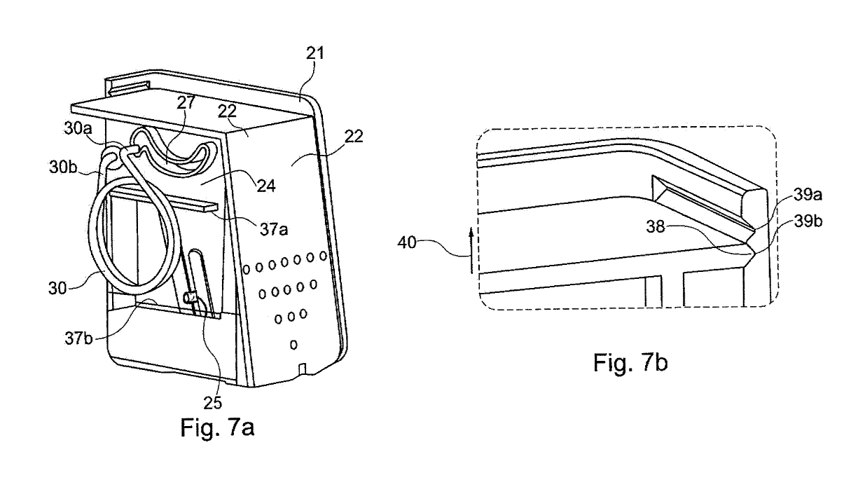

FIG. 7a shows in a perspective and partially open view a further view of the lancing device according to the second embodiment of the invention; and

FIG. 7b shows a detailed view of a setting of the lancing depth for a lancing device according to the second embodiment of the invention.

FIG. 1a shows a perspective view of a lancing device according to a first embodiment of the invention in the assembled state. The lancing device comprises a base body (main body) 1, 2 which is composed of two parts, with an exit opening arranged on the underside for the emergence of a needle tip.

In FIG. 1b, the lancing device according to the first embodiment of the invention is shown in the disassembled state. It can clearly be seen from this diagram that the housing 1 having the exit opening 3 receives the further base body (main body) part 2 which can be pushed in at the top. The base body (main body) part 2 in turn receives a needle holding element 4 which is preferably panel-shaped and can be displaced within the base body (main body) part 2.

The needle holding element 4 has a needle 5 mounted therein, which is preferably permanently encompassed by the needle holding element by means of an injection-moulding process.

The needle 5 is in this case provided at its tip end with a protective cap 6 which may be configured in a sterile manner.

The needle holding element 4 has at its upper end 4a, located opposite the needle tip, a control curve section 7 which is essentially V-shaped, wherein it additionally includes an arresting region arranged on the right-hand side.

The base body (main body) part 2 is provided at the side with a manual actuating element 8, on which there is arranged a release lever 9 for triggering a pressure release process of a spiral-shaped torsional spring 10. The manual actuating element 8 is pivotable and is preferably connected in one piece with the base body (main body) part 2 by means of a film hinge 11.

The torsional spring 10 has a first end 10a and a second end 10b, which run perpendicular to the spiral plane.

Moreover, the base body (main body) part 2 has a slot or a groove 12 which runs perpendicular to the longitudinal axis of the lancing device and which serves to receive one end of the torsional spring.

FIG. 2 shows an enlarged detail view, partially in cross section, of the region of the curve profile of the control curve section. The control curve section 7 has a maximum point 7a at which a first end 10a of the torsional spring, running therein, experiences a maximum excursion relative to the longitudinal direction of the lancing device.

Moreover, the control curve section 7 has an arresting region 7b, the profile of which is designed in such a way that the tensioned torsional spring cannot be moved out of this region towards the V-shaped region of the control curve section without an external force being applied.

In addition, it is also possible to see clearly the horizontally running groove 12 which is arranged on the base body (main body) part 2 and within which the end of the torsional spring also runs, so that, by simultaneously travelling along the control curve section 7 and the groove 12, a displacement in the forward and backward direction of the needle holding element 4 relative to the base body (main body) part 2 automatically takes place.

The spring end is released from the arresting region in the region 7b of the control curve section by means of the release lever 9, which carries out a manually actuated pivoting movement of the manual actuating element 8, and slides into the V-shaped region 7 of the control curve section, resulting in a pressure release of the torsional spring as a result of travelling along the control curve section.

FIGS. 3a-c show the mode of operation of the lancing device with an associated lancet according to the first embodiment of the invention. The lancing device is shown partially in an open and perspective view so as to be able to show the mode of operation more clearly.

FIG. 3a shows the lancing device prior to use. It can clearly be seen from this diagram that the needle holding element 4 is arranged within the base body (main body) 1, 2 with the needle 5 protruding out from the underside and with the protective cap 6 arranged thereon. This needle holding element 4 can be displaced in the forward and backward direction together with the needle 5 within the base body (main body) 1, 2, as illustrated by the arrows 13b and 13c in FIGS. 3b and 3c.

It can likewise clearly be seen in FIGS. 3a-c that the torsional spring 10 is arranged below the needle holding element and is pretensioned under a load in the position shown in FIG. 3a. To this end, the first end 10a of the torsional spring 10 is arranged within an arresting section 7b of the groove-like control curve section 7.

As soon as the manual actuating element 8 is pivoted by means of a hinge 11, as illustrated by the arrow 12, the release lever 9 presses against the first end 10a of the torsional spring 10 in order to move this end out of the arresting region 7b of the control curve section 7 under the effect of force. The pressure on the tensioned spring 10 can then be released by the first end 10a running along the V-shaped curved path of the control curve section 7 until it reaches a maximum point 7a of the control curve section 7, as illustrated in FIG. 3b.

FIG. 3b shows the state of the lancing device during the penetration of the needle tip 5a into the skin of the patient. When the first end 10a of the spring 10 is located at the maximum point 7a of the control curve section, a brief forward displacement of the needle holding element 4 and of the needle 5 integrated therein takes place relative to the base body (main body), so that the needle tip 5a briefly emerges from the exit opening 3 of the base body (main body). This is illustrated by the arrow 13b. This movement is released by a pivoting movement of the manual actuating element, as illustrated by the arrow 13a.

The first end 10a of the spring is then moved in the direction of the arrow 15 into the second portion of the V-shape of the control curve section, as illustrated by the arrow 15. As a result, a rapid retraction of the needle holding element and of the needle tip 5a from its forward position in the base body (main body) 1, 2 takes place.

The second end 10b of the torsional spring 10 is arranged in a stationary manner relative to the base body (main body) part 2.

Such a spiral torsional spring can travel along the groove-like control curve section 7 at different speeds and with varying degrees of force depending on its spring force, so that the spring force also determines the speed of the lancing operation and thus the duration of penetration of the needle tip into the skin.

FIG. 3c shows the lancing device according to the first embodiment of the invention after it has been used. It can clearly be seen in this diagram that the first end 10a of the spring 10 has arrived at the end of the control curve section 7 on the left-hand side and a return movement of the needle holding element and of the needle relative to the base body (main body) has taken place, as illustrated by the arrow 13c.

The lancing device according to the first embodiment of the invention is shown in a partially open perspective view before the lancing operation in FIG. 4a and after the lancing operation in FIG. 4b. A detail view also shows how an adjustment of the penetration depth is possible within the lancing device.

FIG. 4a shows the groove-like slot 12 which is arranged in the base body (main body) part 2 and in which the first end 10a of the spring 10 can engage. At the same time, this first end 10a of the spring 10 engages in the V-shaped profile of the control curve section 7, which is not shown here. The second end 10b of the spring 10 is arranged in a stationary manner relative to the base body (main body) part 2.

The manual actuating element 8 can be pivoted towards the base body (main body), as illustrated by the arrow 16, in order to trigger a lancing operation.

After the end of such a lancing operation, the lancing device is in a state as shown in FIG. 4b. In this state, the first end 10a of the spring has arrived at the end of the groove-like slot 12 on the left-hand side. In this position, the needle holding element and the needle itself are fully retracted into the base body (main body).

A detail 17 shows in an enlarged view how the penetration depth of the needle can easily be adjusted within the lancing device. To this end, the base body (main body) part has preferably triangular protrusions 18 at the side, which can engage in recesses 19a and 19b of complementary shape on the part 1. In this way, a different position of the two parts 1 and 2 is possible by displacing said parts relative to one another in the forward and backward direction. As a result, the needle holding element 4 which is arranged within the base body (main body) part 2 can still be moved forwards, but the needle arranged therein with the needle tip 5a can no longer exit from the base body (main body) part 1 by the same distance.

Depending on how close or distant the base body (main body) part 2 is arranged relative to the base body (main body) part 1 at their undersides, the needle tip 5a can exit to a greater or lesser extent from the exit opening 3 of the base body (main body) part 1. This makes it possible in a simple manner to set the penetration depth of the needle tip into the skin of the patient.

FIG. 5a shows a perspective view of a lancing device according to the second embodiment of the invention in the assembled state. The lancing device comprises a first base body (main body) part 21 and a second base body (main body) part 22 and also an exit opening 23 arranged on the underside for the emergence of the needle tip.

In FIG. 5b, the lancing device is shown in a perspective view and in the disassembled state. The lancing device has on the second base body (main body) part 22 a needle holding element 24 with a needle 25 arranged therein, which has a needle tip 25a.

A release lever 29 which is attached to a manual actuating element 28, which is in turn arranged on the base body (main body) part 22 by means of a hinge preferably designed as a film hinge 31, serves to move a first end 30a of a spiral torsional spring out of an arrested position within a control curve section 27.

The torsional spring 30 has, in addition to the first end 30a, a second end 30b which is connected in a stationary manner to the base body (main body) 22.

FIGS. 6a-6c show, in a perspective and partially open view, different functional states of the lancing device according to the second embodiment of the invention. The diagram shown in FIG. 6a shows the lancing device before use, in FIG. 6b the lancing device is shown during the penetration of the needle tip 25a into the skin of a patient, and in FIG. 6c the lancing device is shown after use.

By means of a pivoting movement 32 of the manual actuating element 28, the release lever 29 is in turn pressed against the first end 30a of the spring 30 in order to release the latter in the direction of the arrow 34 from an arrested position in an arresting region 27b of the control curve section 27. A pressure release of the pretensioned spring 30 then takes place by travelling along the control curve section 27.

While travelling along the control curve section 27, after the manual actuating element 28 has been pivoted as illustrated by the arrow 33a, a low point or a maximum excursion point 27a of the arc-shaped profile of the control curve section 27 is run through, during which a penetration of the needle tip 25a into skin (not shown here) takes place. During this displacement movement, which is illustrated by the arrow 33b, the needle 25 moves out of the base body (main body) part 22 and penetrates into the skin. During this, the needle tip points in a direction 36b, whereas before the actual lancing operation the needle tip 25a is oriented in the direction of the arrow 36a, as shown in FIG. 6a. Accordingly, by comparing the three arrows 36a, 36b and 36c, it can clearly be seen from these figures that the needle 25 carries out not only a forward and backward displacement movement, as illustrated by the arrows 33b and 33c, during the lancing operation, but also a slight pivoting movement. In this case, it is advantageous that the control curve section 27 is arranged at the upper end 24a of the needle holding element 24, i.e. at that end located opposite the needle tip 25a, since as a result a desired slight pivoting movement of the needle tip 25a takes place during the forward and backward displacement thereof, due to the long lever arm of the needle 25.

After the actual penetration of the needle tip 25a into the skin of the patient, the end 30a of the spring 30 arranged at the top end of the needle is moved upwards to the left in the direction of the arrow 35 in order to reach its end position, as shown in FIG. 6c.

Compared to the lancing device according to the first embodiment, the lancing device according to the second embodiment is equipped with a needle which can be displaced relative to the needle holding element 24 and is not arranged in a stationary manner relative to a needle holding element 4. Accordingly, in the lancing device according to the second embodiment, it is not necessary for the needle holding element 24 to be able to be displaced relative to the base body (main body) part 21, 22, since the needle itself is displaced in the forward and backward direction.

FIG. 7a shows a perspective, partially open view of the lancing device according to the second embodiment of the invention. It can clearly be seen from this diagram that the needle 25, which is designed as a flat element, is attached to the needle holding element 24 at the top and at the bottom by being guided through slots 37a and 37b. This allows a fixing of the needle 25 in the Z direction in a simple manner, whereas it can still be displaced or moved in the X and Y direction so as to be able to carry out the lancing operation.

The spiral torsional spring 30 is arranged with its second end 30b in a stationary manner on the base body (main body) part 22 and is arranged with its first end 30a such that it can move within the control curve section 27.

The movable end of the torsional spring 30 is received in a cutout of the end of the needle 25, as a result of which the needle is moved by the spring after the release operation has taken place. Moreover, the needle is guided via an elongate hole which is mounted in a displaceable and rotatable manner on a guide pin of the base body (main body) part 22. This elongate hole or the slot allows a guidance of the needle during its forward and backward movement and also its pivoting movement. In this case, the needle is designed as a knife-shaped part in order to allow a cutting movement with the blade thereof to enable a larger quantity of blood to be taken.

FIG. 7b shows a detail of the lancing device according to the second embodiment. Once again, a preferably triangular protrusion 38 is arranged on the side of the base body (main body) part 22 in order to be able to engage in corresponding recesses 39a and 39b of complementary shape within the base body (main body) part 21. By a displacement of the two parts relative to one another, as illustrated by the arrow 40, a distance of the underside of the base body (main body) part 22 from the underside of the base body (main body) part 21 can be set to different degrees, resulting in a different degree of excursion of the needle tip of the needle out of the base body (main body) part 21. As a result, the penetration depth into the skin of a patient is predetermined and can be varied in a simple manner.

The needle 25 is held and guided within the base body (main body) by two slots which run perpendicular to the longitudinal direction of the lancing aid device, and thus allows in a simple manner a fixing of the needle in the Z direction. A demoulding of the needle guide pin is thus also possible in a simple manner during the injection-moulding process.

In the first embodiment, the needles may be designed as round needles, i.e. as needles with a round cross section, or with any other cross section. In the second embodiment, these are needles which are designed as flat knives, so that these are sharpened in a blade-like manner on their cutting edge in order to allow a larger cut in the skin for the purpose of taking a greater quantity of blood.

Instead of being attached by means of film hinges, all of the pivoting elements may alternatively be designed as displaceable elements. These would then not be connected in one piece with the base body (main body).

The needles may have protective caps which serve as sterile protection and have to be twisted off by hand in a separate operating step prior to actuation of the lancing aid.

All of the features disclosed in the application documents are claimed as essential to the invention in so far as they are novel individually or in combination with respect to the prior art.

### LIST OF REFERENCES

1, 2 base body (main body)
3, 23 exit opening
4 needle holding element
4a end of the needle holding element
5 needle
5a needle tip
6 protective cap
7 control curve section
7a maximum point
7b arresting section
8 manual actuating element
9 release lever
10 torsional spring
10a, 10b ends of the torsional springs
11 film hinge
12 groove
13b, 13c arrows
15 arrow
16 arrow
19a, 19b recesses
21, 22 base body (main body) part
24 needle holding element
24a end
25 needle
25a needle tip
27 control curve section
27a excursion point
27b arresting region
28 manual actuating element
29 release lever
30 spring
30a, 30b end
31 film hinge
32 pivoting movement
33a, 33b, 33c arrow
34 arrow
36a arrow
36b direction
37a, 37b slots
39a, 39b recesses
40 arrow

1. A lancing device for taking blood for medical examinations, comprising a base body, at least one needle which is arranged therein and which can be extended outwards with a tip end, a needle holder which at least partially encompasses the needle, a manual actuator for releasing a displacement movement of the needle, a control curve section, and a torsional spring that engages in the control curve section, wherein the control curve section and the torsional spring are arranged with respect to the needle holder in order to transform a radial movement of first and second ends of the torsional spring into translational displacement movement of the needle.

2. The lancing device according to claim 1, wherein the needle holder is configured to be displaced together with the needle within the base body.

3. The lancing device according to claim 1, wherein the needle holder is arranged in a stationary manner in the base body and the needle can be displaced relative to the needle holder and the base body.

4. The lancing device according to claim 1, wherein the translational displacement movement of the needle is coupled to a pivoting movement of the needle in the cutting direction.

5. The lancing device according to claim 1, wherein:
the control curve section comprises a curved groove in the region of an end of the needle holder opposite the tip end of the needle;
the torsional spring has first and second ends; and
the first end of the torsional spring is configured to slide in the curved groove of the control curve section and the second end of the torsional spring is connected in a substantially stationary manner to the base body.

6. The lancing device according to claim 5, wherein the curved groove of the control curve section comprises a first region configured to allow an arresting of the first end of the torsional spring, and a second region configured with a substantially arc-shaped profile.

7. The lancing device according to claim 6, wherein the first region of the curved groove of the control curve section is connected to a guide groove section which runs substantially perpendicular to the direction of displacement of the needle holder.

8. The lancing device according to claim 6, wherein the substantially arc-shaped profile has a maximum point oriented towards the end of the needle holder opposite the tip end of the needle.

9. The lancing device according to claim 6, wherein the substantially arc-shaped profile has a maximum point oriented towards the tip end of the needle within the needle holder.

10. The lancing device according to claim 6, wherein the manual actuator is pivotally connected to the base body and further comprises a release lever configured to move the first end of the torsional spring from its arrested position in the first region of the control curve section.

11. The lancing device according to claim 2, wherein the first end of the torsional spring engages in a groove which runs substantially perpendicular to the direction of displacement of the needle holder and which is arranged in a stationary manner with respect to the base body.

12. The lancing device according to claim 1, wherein
the base body is composed of first and second housing members; and
holder and the torsional spring are arranged substantially on the first member.

13. The lancing device according to claim 12, wherein the first housing member is movably attached to the second housing member and configured to be displaced relative to the second housing member in a latching manner.

14. A lancing device for taking blood for medical examinations, comprising:
a base body;
at least one needle having a tip end and configured within the base body to extend outwards with the tip end;
a needle holder at least partially encompassing the needle;
a manual actuator movable connected to the base body and configured to release a displacement movement of the needle and the needle holder relative to the base body;
a control curve section; and
a torsional spring configured to engage the control curve section, wherein the control curve section and the torsional spring are arranged with respect to the needle holder in order to transform a radial movement of ends of the torsional spring into displacement movement of the needle.

15. The lancing device according to claim 14, wherein the displacement movement of the needle is coupled to a pivoting movement of the needle in the cutting direction.

16. The lancing device according to claim 14, wherein:
the control curve section comprises a curved groove in the region of an end of the needle holder opposite the tip end of the needle; and
the first end of the torsional spring is configured to slide in the curved groove of the control curve section which is arranged in a stationary manner with respect to the base body.

17. The lancing device according to claim 16, wherein the curved groove of the control curve section comprises a first region configured to allow an arresting of the first end of the torsional spring and a second region configured with a substantially arc-shaped profile.

18. The lancing device according to claim 17, wherein the first region of the curved groove of the control curve section is connected to a guide groove section which runs substantially perpendicular to the direction of displacement of the needle holder.

19. The lancing device according to claim 17, wherein the manual actuator is pivotally connected to the base body and further comprises a release lever configured to move the first end of the torsional spring from its arrested position in the first region of the control curve section.

20. A lancing device for taking blood for medical examinations, comprising:
a base body;
at least one needle having a tip end and configured within the base body to extend outwards with the tip end;
a needle holder arranged in a stationary manner in the base body and at least partially encompassing the needle;
a manual actuator movable connected to the base body and configured to release a displacement movement of the needle relative to the needle holder and the base body;
a control curve section; and
a torsional spring configured to engage the control curve section, wherein the control curve section and the torsional spring are arranged with respect to the needle holder in order to transform a radial movement of ends of the torsional spring into translational displacement movement of the needle.

21. The lancing device according to claim 20, wherein the displacement movement of the needle is coupled to a pivoting movement of the needle in the cutting direction.

22. The lancing device according to claim 20, wherein:
the control curve section comprises a curved groove in the region of an end of the needle holder opposite the tip end of the needle; and
the first end of the torsional spring is configured to slide in the curved groove of the control curve section which is arranged in a stationary manner with respect to the base body.

23. The lancing device according to claim 22, wherein the curved groove of the control curve section comprises a first region configured to allow an arresting of the first end of the torsional spring and a second region configured with a substantially arc-shaped profile.

24. The lancing device according to claim 23, wherein the first region of the curved groove of the control curve section is connected to a guide groove section which runs substantially perpendicular to the direction of displacement of the needle holder.

25. The lancing device according to claim 23, wherein the manual actuator is pivotally connected to the base body and further comprises a release lever configured to move the first end of the torsional spring from its arrested position in the first region of the control curve section.
